(19) United States
(12) Patent Application Publication
(10) Pub. No.: US 2023/0285631 A1
(43) Pub. Date: Sep. 14, 2023

(54) COATING OF A STRUCTURED IMPLANT SURFACE

(71) Applicant: WALDEMAR LINK GmbH & Co. KG, Hamburg (DE)

(72) Inventors: Helmut D. LINK, Hamburg (DE); Sebastian SPATH, Hamburg (DE); Richard CSASZAR, Bad Segeberg (DE)

(73) Assignee: WALDEMAR LINK GmbH & Co. KG, Hamburg (DE)

(21) Appl. No.: 18/006,154

(22) PCT Filed: Aug. 9, 2021
(86) PCT No.: PCT/EP2021/072161

§ 371 Date: Jan. 20, 2023

(30) Foreign Application Priority Data
Aug. 10, 2020 (EP) 20190262.4

(51) Int. Cl. A61L 27/30; A61L 27/06; A61L 27/04; A61L 27/56; A61L 27/54

(52) U.S. Cl. CPC A61L 27/306; A61L 27/06; A61L 27/047; A61L 27/56; A61L 27/54; A61L 2300/104; A61L 2300/404; A61L 2300/606; A61L 2400/18; A61L 2420/02; A61L 2430/02; A61F 2002/3092

(57) ABSTRACT

An implant component which comprises a solid material region and a surface structure connected to the solid material region is disclosed. A coating is provided on the surface structure, said coating comprising, in addition to an At % proportion of Ti as a main component, at least one further coating component, wherein one of the at least one further coating components is silver having an At % proportion of 15-25 At %. The surface structure here comprises undercuts which are coated with said coating.

### CROSS-REFERENCE TO RELATED APPLICATIONS

This application is a National Stage under 35 U.S.C. 371 of International Patent Application No. PCT/EP2021/072161, filed Aug. 9, 2021, which claims priority from European Patent Application No. 20190262.4, filed Aug. 10, 2020; the disclosures of all of which are incorporated herein by reference in their entirety.

### TECHNICAL FIELD

The present invention relates to an antimicrobial implant component with a coated surface structure and to a method for coating such an implant component provided with a surface structure with an antimicrobial coating.

### BACKGROUND

In the integration of an implant component in the anatomical surroundings of an implant site of a patient, the implant surface plays an essential role. Mainly for this reason, it is attempted in numerous ways to provide or modify the implant surface in such a way that an implant component can successfully perform its task.

A possible purpose for the selection of a certain implant surface can be, for example, that the implant surface promotes the ingrowth of bone tissue or soft tissue at the implantation site. In particular, a variety of approaches exist for this purpose, in order to influence the properties of the implant surface. Those include, for example, the selection of the material of which the implant surface consists, or the constitution of the surface structure which comes in contact with the tissue after the implantation.

One approach to implementing an implant surface consists in providing the material of the implant body with a defined surface structure in order to thus increase, for example, the implant surface and thereby the contact surface with the surrounding tissue. For this purpose, the implant surface itself or the implant surface with the application of further material is structured. An implant surface thus generated can promote ingrowth of the tissue and thus the integration of an implant component.

In addition to the generation of a surface structure, an implant surface can be modified by the application of a coating, in order to support the functionality of the implant surface to support the respective treatment goal. However, if the implant surface to be coated is a structured surface, this results in requirements for the coating process, as before. Those include, for example, the homogeneity or the uniform thickness of the coating. These requirements are in particular due to edges, points and undercuts of the surface structure.

### SUMMARY

Thus, one aim of the present disclosure was to provide an implant component with a surface structure, wherein the surface structure comprises undercuts and the surface structure including the undercuts is coated with a coating. In addition, it was an aim of the present invention to provide a technical method for generating a coating on this surface structure. A further aim was that the coating generated by the method is formed as continuously and homogeneously as possible on the surface and has an antimicrobial effect.

As a result of one or more of the problems resulting from the prior art, the claimed invention is defined by the attached independent claims. In addition, the associated dependent claims define other embodiments.

In particular, the inventors of the claimed subject matter were faced with the observation that an antimicrobial effect of coatings on implants with a surface structure but without undercuts can at least not necessarily be transferred to a surface structure with undercuts. In particular, it was noted that the antimicrobial coating substantially loses the sought effect.

In order to counteract this, an implant component which has a solid material region and a surface structure connected to the solid material region is disclosed. On the surface structure, a coating is provided, which, in addition to an At % proportion of Ti, comprises at least one further coating component as a main component, wherein one of the at least one further coating components is silver (Ag) having an At % proportion of 15-25 At %. Here, the surface structure comprises undercuts which are coated with said coating.

Tests of the inventors have shown that the use of a conventionally used antimicrobial coating on an implant component with surface structure does not lead to the desired reduction of pathogens. It is assumed that in particular undercuts of the surface structure contribute to this effect in that they affect the mode of action of the antimicrobial coating.

In spite of this experience, it was nevertheless surprisingly determined by further tests that a silver proportion within the above-defined region leads to an antimicrobial effect. It was assumed that this can be attributed to an increased effect of the coating in the region of the undercuts. In other words, the above-defined coating presumably has the effect that the antimicrobial effect is achieved at least on a higher proportion of the surface of the implant component, which is increased by the surface structure, and namely in particular in the case of undercuts.

The coating for the surface structure comprises titanium as a main component, since this material was proven to be effective in particular for the ingrowth of bone tissue. Depending on the application case, titanium can be supplemented with further coating components in addition to silver.

It was determined that, in the case of the At % proportion of silver, in particular a silver proportion sufficient for an antimicrobial effect, is present not only on the regions visible in a top view but also on the regions of the surface structure, which are covered in a top view, i.e., on the undercuts.

An undercut is here understood to be a region of the surface structure which is covered in a top view onto an implant component since it is situated behind the visible portion of the surface structure. An undercut can, for example, be formed by an angled recess or an angled protrusion.

Such undercuts are formed by the generation of a surface structure on the solid material region of the implant component. This can occur by a modification of the solid material by ablation of material on its surface. However, additionally or alternatively, the surface structure is constructively generated on the solid material. In other words, material is applied to the surface in order to construct a surface structure.

The surface structure is provided with its increased surface in particular for the ingrowth of bone tissue. This is advantageous in particular in the case of implant components which are to be integrated in the bone tissue in order to thus form a rigid connection to the bone tissue of an implantation site. For example, such a connection is advantageous in the case of joint implants, joint stiffeners or bone replacement parts.

The undercuts of the surface structure can be at least partially formed by an open-pore structure.

In other words, the surface structure connected to the solid material is porous. The surface structure thus comprises pores which are open and connected to one another. Correspondingly, it becomes possible for bone tissue to grow into this open-pore structure. The pores can consequently complement one another to form cavities or connection channels.

On the one hand, such a structure, to some extent, resembles a spongy bone structure and promotes bone ingrowth, and, on the other hand, it has the advantage that it serves as a kind of interface between the usually very stiff solid material of the implant component and the substantially softer bone tissue compared thereto. The surface structure thus has a structurally caused lower stiffness than the solid material of the implant component. This too can lead above all to a long-term improved integration of the implant component.

With regard to the mechanical resistance of the coating, the silver proportion in fact lowers the hardness of the coating but, on the other hand, the ductility of the coating is increased. This is advantageous in particular in the case of a coated porous surface structure, since said surface structure, as described above, is softer and, in particular, it can more simply undergo elastic and/or plastic deformation during the implantation. The coating can at least adapt to such a deformation to a greater extent due to the ductility.

In addition, it can be prevented that the solid material of the implant component located under the coating comes in contact with body tissue of a patient and in the process potentially leads to hypersensitivity. This is in particular the case if the surface structure is carved out or if an applied surface structure does not completely cover the solid material of the implant component. The coating then ensures in this region that the surrounding tissue does not come in contact with the solid material.

One possibility for generating an open-pore surface structure is the use of a plasma spray coating, in particular a titanium plasma spray coating. The use of a titanium plasma spray coating has the additional advantage that the coating of the surface structure thus generated forms a more robust connection.

Additionally, or alternatively, the open-pore structure can comprise substantially regularly arranged unit cells, wherein the unit cells are formed by tetrapod-like basic elements.

Due to the regularity of this open-pore structure, the stiffness and the pore size or pore width of the surface structure can be established very well in advance. Correspondingly, advantages for bone ingrowth as well as advantages with regard to the stiffness behavior can be implemented. For this purpose, unit cells formed by tetrapod-like basic elements have been shown to be particularly suitable.

The open-pore structure can have a porosity of 10 to 80% and/or a pore width of 45 to 1000 μm.

These properties relate in particular to an uncoated open-pore structure. They promote a coating of the pore inner surfaces and thus also of the undercuts with a sufficiently high silver proportion. In other words, the penetration of the silver ions during the coating process into the surface structure is supported. These values also apply to surface structures which are not necessarily porous but comprise recesses forming undercuts.

A pore of the open-core structure, depending on its already aforementioned formation, can comprise pores of substantially identical shape and identical size and/or pores of less identical shape and having different sizes. In the last case, the pore width relates to the widest cross section. Here, the ratio between the widest and the narrowest cross section of a pore is at most 4:1 or 2:1. In general, a ratio of approximately 1 is considered to be particularly advantageous.

The surface structure on the solid material can be formed with a thickness of up to 4 mm, 3 mm, 2.5 mm, 2 mm, 1.5 mm, 1 mm or 0.5 mm.

Thereby, a sufficiently rigid integration of the implant component in the bone tissue as well as a satisfactory supply of the bone tissue is achieved.

In particular, the silver content of the coating is at least 18 At %, 20 At % or 22 At % and at most 23 At %, 24 At % or 25 At %.

In these regions, the silver proportion in the coating is sufficiently high so that, in the case of the implant component, an antimicrobial effect of the surface structure appears.

Particularly, the coating comprises an At % proportion of N and/or an At % proportion of Nb as a further coating component.

The use of N and/or Nb as coating components enables an adaptation of the properties of the coating.

Here, in combination with the silver proportion, the result is not only an antimicrobial effect but also the possibility of setting the properties of the coating by providing one or both of these coating components. In addition, these coating components prevent allergic reactions of patients to the material of the solid material region of the implant component.

The coating can be substantially in the form of a stoichiometric coating, independently of the coating components used. Such a coating, together with the silver proportion, forms a particularly uniform inert layer with antimicrobial effect and thus prevents both a hypersensitivity and an infection after implantation of the implant.

By means of an At % proportion of N, in addition to the main component Ti and the At % proportion of Ag, in particular a titanium nitrate coating with a silver proportion (TiN—Ag) can be generated.

In particular, nitride coatings can be used in order to improve the abrasion properties of implant components. Therefore, they are used, for example, for the bearing surfaces of joint implants but also on other surfaces which are exposed to a friction force, such as, for example, fastening elements, clamping surfaces or implant surfaces, which are exposed in particular during implantation to a relative movement in contact with another implant component or bone tissue.

Due to its resistance, the ceramic titanium nitride coating with a silver proportion, i.e., a titanium nitride-silver coating, is particularly suitable for structural implants which support or replace parts of the skeleton, when they have been introduced into the body of a patient. This includes, for example, an implant component of a joint implant, of a vertebral column implant or else of a bone implant which replaces at least a portion of a bone.

By means of an At % proportion of Nb, in addition to the main component Ti and the At % proportion of Ag, in particular a titanium niobium coating with a silver proportion (TiNb—Ag), can be generated.

The titanium-niobium coating, like titanium, has a high degree of biocompatibility. In particular, the coating has a lower degree of hardness, and it is more ductile than a TiN coating. Consequently, it can adapt to elastic and plastic deformations which occur when an implant is used to a greater degree than the TiN—Ag coating. Like the latter, it also effectively protects against an occurrence of a hypersensitivity of the surrounding tissue.

Moreover, it is possible to generate, in addition to the main component Ti and the At % proportion of Ag, a titanium nitride-niobium coating with a silver proportion (TiNbN—Ag) by providing an At % proportion of N and an At % proportion of Nb.

Such a coating is resistant to abrasion and also sufficiently ductile to withstand elastic as well as minor plastic deformations as they occur, for example, during the adaptation of bone plates or spondylodesis rods. In terms of its properties, it is thus substantially between the properties of the TiN—Ag coating and the TiNb—Ag coating.

Moreover, it is possible to combine the above coating types in order to set the properties of the coating.

The coating of the implant can be a PVD coating. This means that the coating is applied by means of a physical gas phase deposition. Due to the gas phase deposition, the ionized coating components particularly effectively reach any possible undercuts or pores of the surface structure.

In the above coatings, the At % proportion of silver in the coating can be in the form of islets.

With regard to the aforementioned coating, this means, for example, that Ag islets are surrounded by Ti, TiN, TiNb and/or TiNbN.

Thereby, the Ag proportion can particularly effectively develop its infection-inhibiting effect. In order to achieve this adjacent buildup of the coating on the surface and thus a direct contact with the tissue or fluids of the patient, the coating components are simultaneously applied at least during part of the coating process. Due to this simultaneous application of the coating components, the coating components moreover are substantially uniformly distributed on the implant surface.

Particularly, the coating has a thickness of 1-6 μm, 2.5-6 μm or 3.5-5.5 μm.

In these regions, a stable, continuous, and resilient coating on the surface structure and on the solid material of the implant component can be achieved, said solid material possibly being exposed under the surface structure, as described above.

Generally speaking, a thicker coating is advantageous. However, a thickness exceeding the aforementioned values does not bring about any noteworthy advantages but instead can even promote inhomogeneity and delamination of the coating. It is assumed that, in the case of the indicated thicknesses, the mechanical resistance of the coating is also due to the fact that none of the sections which is continuous in the thickness direction is formed from silver. In other words, the three-dimensionally heterogeneous structure of the silver-containing titanium coating results in a coating of the titanium or of the titanium compound, which is in principle continuous.

Furthermore, the present disclosure relates to a method for applying a coating onto an implant component, wherein the method includes the following steps: first, an implant component, in particular an implant component according to any one of the preceding claims, is provided, wherein the implant component is formed with a solid material region and with a surface structure connected to the solid material region and the surface structure comprises undercuts. The implant component is introduced into a coating chamber. Moreover, for the application of the coating at least one target made of a metallic material is provided, which comprises at least one of the coating components. Before the start of the coating process, the coating chamber is closed and locked, and an atmosphere with a pressure of 0.001 to 0.01 mbar is provided. The atmosphere can here comprise a coating component. After the providing of the atmosphere, the ignition of an electric arc for evaporating the metallic material of the at least one target, and the coating of the surface structure (and possibly of an exposed region of the solid material under the surface structure) with the evaporated metallic material of the at least one target occur.

This method enables a simultaneous and above all continuous coating of the surface structure inside the coating chamber. Here, by means of the simultaneous coating and the silver proportion, it is ensured that, on the surface of the coating, silver is exposed, and thus the infection-inhibiting effect of the coating can be developed. This also applies to any possible undercuts or pores. In addition, by a selection of the number of the respective targets of the coating components, the At % proportion can be set at least in its order of magnitude to the desired composition of the coating.

Alternatively, or additionally to the setting of the composition of the coating via the number of the respective targets, at least one target comprising a predetermined ratio of at least two coating components can be provided. In particular, the at least one target comprises silver, titanium and/or niobium.

As an alternative to the above pressure range, the pressure of the atmosphere for the coating process can also be set to a pressure of 0.003 mbar or 0.005 mbar to 0.1 mbar, 0.05 mbar, 0.02 mbar, 0.015 mbar or 0.01 mbar. The higher pressure range has the advantage that the coating components thereby reach the undercuts or pores to a greater extent and thus it ensures a greater and more uniform coating thickness.

Moreover, in the context of the provision of the implant component, the surface structure can be formed by particles which are melted together, which are applied by means of a plasma spraying method, in particular by means of the titanium plasma spraying method, onto the solid material of the implant component.

Such a surface structure, due to its undercuts and/or porosity, promotes the ingrowth of bone tissue and thus, a stable integration of the implant component.

Particularly, the surface structure can be constructed from unit cells and formed by means of a layer melting method, in particular an electron beam layer melting method.

The surface structure also promotes the ingrowth of bone tissue and, in addition, as described above, it forms a substantially regular surface structure, the properties of which can be set by the size of the unit cells or of the tetrapod-like basic elements which are the basis of said unit cells. In addition, the regularity of the structure favors a uniform distribution of the surface structure.

### DETAILED DESCRIPTION

In the context of the present invention, a coating is understood to mean a coating applied by a technical method. Examples of such technical methods are gas phase deposition (CVD—Chemical Vapor Deposition or PVD—Physical Vapor Deposition) or else plasma spraying methods such as, in particular, the already aforementioned titanium plasma spraying method.

As described above, a coating according to the invention includes titanium and silver, as well as up to two further coating components. For the sake of simplicity, such a coating is referred to below as titanium-silver coating, it being understood that said coating can also include further coating components. In addition, it is noted that the coating by the technical coating process can contain up to a proportion of 3%, 2% or 1% contaminants in the form of other elements or compounds. This also applies, in particular, to a coating which consists of certain coating components.

The application of the coating here occurs on a surface structure of the implant component. One method with which such a surface structure can be generated is a plasma spraying method. In the plasma spraying method, substantially in a vacuum, a plasma beam is generated, which is supplied with metal particles which melt in the high-energy beam and are accelerated. Subsequently, the molten metal particles strike the surface of the implant component and connect to it to a greater extent. The metal particles can comprise titanium or a titanium alloy or substantially consist thereof.

A surface structure generated by the plasma spraying method can have a roughness (Ra) in the range of 20 μm to 80 μm. In addition, a surface structure generated with this method comprises in particular undercuts.

It was noted that these undercuts are not sufficiently coated with a conventionally used antimicrobial coating for the desired effect to develop. It has then been assumed that this is in particular due to a lower silver proportion in this region.

In addition, a surface structure generated with a plasma method is can be porous. The porosity can be in a range of 10% to 60%, or in a range of 15% to 60%, or in a range of 20% to 50% (measured according to ASTM F1854).

In these ranges, in addition, a sufficient pore width for the ingrowth of tissue, in particular of bone tissue, can be provided. Thus, the surface structure in particular has pore widths in a range of approximately 45 μm to 80 μm, or in a range of 50 μm to 70 μm. The average pore width of all the pores can also be in this range (arithmetic average).

Another method generates the surface structure by means of an additive method. In particular, layer melting methods are suitable for this purpose, such as, for example, the already aforementioned electron beam layer melting method. The advantage of additive methods is that the surface structure can be formed in a defined and regular manner on the implant component. In the case of such an additive method, the solid material and the surface structure of the implant component can be produced substantially in the same production step.

As presented above, the surface structure can comprise unit cells, from which it is constructed. The unit cells in turn can be constructed from at least one element type, in particular from exactly one basic element type such as, for example, a tetrapod-like basic element. In this regard, reference is made in particular to the porous structures disclosed in patent application WO 2017/005514 A1, which is included hereby by reference.

One advantage of a porous structure produced with an additive method is that the porosity, the pore width, the pore size and/or the pore shape can be produced in a regular manner. In addition, the formation of the surface structure allows for predetermined mechanical properties such as, for example, a (direction-dependent) elasticity. Thus, the surface structure can be arranged in particular for a certain tissue such as, for example, bone tissue or connective tissue.

By an additive method, a predefined and in particular also higher porosity of the surface structure can also be produced. Thus, the porosity can be 50% to 80%, or 65% to 75% (measured according to ASTM F1854).

Furthermore, in an additive method, the sufficient pore width for the ingrowth of tissue, in particular of bone tissue, can be provided in a simple manner. Thus, the surface structure has in particular pore widths in a range of approximately 100 μm to 1000 μm, or in a range of 300 μm to 900 μm, or in a range of 500 μm to 800 μm. Due to the regularity of the structure, the average pore width is also substantially predetermined and deviates only little from the target size as a result of the production process (for example, the arithmetic average can deviate by less than 50 μm). In other words, the variance is lower in additive methods than in other methods such as, for example, in the aforementioned plasma spraying methods.

The pore sizes generated in the above methods can have a ratio between widest and smallest pore width which is less than 4:1, 3:1, or 2:1.

The coating of the surface structure comprises one, two or three layers of the coating, in which the silver is in embedded form. In particular, the silver is in the form of silver islets (silver agglomerates), i.e., silver or silver atoms are arranged next to the mesh of the remaining coating components. Due to the size of the silver atoms, it is assumed that only a small proportion of the silver, if any at all, is interstitially arranged in the mesh.

In particular, it was observed that the silver is in the form of silver agglomerates in the titanium-silver coating.

In other words, the silver is present in the mesh of the coating but not integrated therein. The silver agglomerates can be present in a range of 1 μm to 50 μm, or in a range of 5 μm to 30 μm.

Moreover, it is assumed that the efficacy of the silver arises in particular because the silver transitions in the implanted state of the implant component in contact with the body fluid by local unit formation into the ionic state and thus develops its antimicrobial effect. That this local unit formation can occur is very effectively made possible by an arrangement of these islets on the surface of the coating. This arrangement is achieved due to an at least partially simultaneous coating of the implant component with titanium nitride and silver.

The coating has an infection-inhibiting effect due to its antimicrobial properties. It is assumed that the present silver proportion of the coating disturbs the formation of a biofilm which these bacteria develop. Due to this disturbance, the protection brought about by this biofilm is then no longer sufficient for the bacteria, so that they can be attacked a greater extent by the immune system of patients or else by active ingredients.

Moreover, it was observed that the silver in ionic form can be dissolved out of this coating. It is assumed that these silver particles which are ionized on the surface of the coating, in the immediate surroundings of the implant component form an action zone (“inhibition zone”) in which they develop an antimicrobial effect. Consequently, not only is it possible with the coating to prevent an infection propagating directly from the surface of the implant component, but in particular it is also possible to prevent an infection in patient tissue adjoining the implant component.

The At % proportion of silver can be lower than the At % proportion of titanium. In other words, it is not necessary for a stoichiometric distribution to be present. The distribution can be superstoichiometric or substoichiometric.

The silver proportion, together with the proportion of titanium and possible further coating components, leads to a change of the mechanical properties compared to a coating without silver proportion, in addition to the aforementioned antimicrobial effect. In particular, the coating becomes softer and more ductile due to this structure.

It is assumed that, due to a ductility associated therewith, the mechanical resistance or strength of the coating furthermore is still sufficient to withstand the mechanical influences occurring during an implantation of the implant component. Such mechanical influences arise, for example during the generation of a press fit of an implant component in the bone tissue, due to contact of an implant component with a fastening element, such as, for example, during the screwing in of bone screws for fastening a plate, or during the mounting with another implant component, such as, for example, in a spondylodesis structure.

For this reason, the present coating is particularly suitable for implant components which, after implantation, support the skeleton of a patient or replace portions of this skeleton. In such implant components, a mechanical loading of the coating generally occurs during the implantation and the assembly of multiple implant components. After the implantation, in particular due to daily loading of the implant in the body of a patient, tensions and expansions in the coating occur due to elastic deformations. Here too, the present coating is advantageous since it withstands said deformations without losing its protective effect.

As described above, the coating components can be selected so that the coating is mainly suitable for implant components, wherein abrasion occurs mainly during the implantation of the implant component and/or the assembly of multiple components. It has been noted that, for this purpose, a thickness of the coating of less than 10 μm, in particular of 2.5-6 μm, 3.5-5.5 μm, or approximately 4.5 μm is sufficient. This also applies to the above-described TiNb—Ag coating which has a particularly high adaptability to a deformation of the surface structure.

Furthermore, in the present coating, due to the silver proportion, a difference of its material properties, in particular of the elasticity, compared to the underlying material of the surface structure can be partially reduced. A sufficient mechanical resistance and adhesion of the coating can also be promoted thereby.

In summary, the present coating for the surface structure of an implant component thus has both advantageous antimicrobial properties and advantageous mechanical properties which are useful for an implant component which is coated at least in sections with this coating.

Such a coating can be produced by the already aforementioned physical gas deposition (PVD method). Before the introduction into the coating chamber, the implant component provided with the coating, including its surface structure, can be cleaned with water.

The implant component is then introduced into the coating chamber which is subsequently evacuated. For the subsequent processes, the implant component can be heated to 400 to 600° C., in order to improve the motility of ions on the surface structure of the implant component and thus achieve a better adhesion and distribution of the coating on the surface structure and on a surface structure of the solid material, which is to be coated and possibly situated beneath.

Moreover, it is possible to carry out a cleaning of the implant surface by ion etching. Here, the implant component is bombarded with ions (for example, titanium ions, argon ions) under an inert atmosphere, in particular an argon atmosphere, in order to remove an oxide layer possibly present on the surface of the uncoated surface structure. A better adhesion of the coating on the surface structure of the implant component is also achieved thereby.

After this optional cleaning, the application of the coating onto the implant component under an atmosphere occurs. As for the atmosphere, in the case of the use of nitrogen as coating component, an atmosphere containing at least nitrogen is used. Otherwise, the coating occurs under an atmosphere substantially consisting of an inert gas.

As previously described above, this coating, depending on its desired composition, can be carried out with at least one silver target, at least one titanium target and at least one further target with a further coating component. Likewise, it is possible to use one or more targets comprising the At % proportions of silver, titanium and possibly further coating components provided for the coating. Here, the composition of the coating is consequently determined in particular by the composition of the at least one target.

In order to bring about a scattering of the evaporated target material on gas particles in the coating chamber so that they reach the surfaces of undercuts or pores to a greater extent, the coating occurs under the aforementioned low-pressure regions.

Once the desired atmosphere is set, the evaporation process of the at least one target starts. Particularly, an electric arc is used for this purpose, which dissolves material out of the targets due to a strong current by means of an electrical discharge and converts it into the gas phase. During this discharge, in particular voltages in a range of 15-30 V, or in a range of 20-25 V as well as currents in a range of 40-70 A are used. However, it is understandable to a person skilled in the art that other methods for evaporating the targets can be used, such as, for example, thermal evaporation, electron beam evaporation or laser beam evaporation.

At least during part of the coating process, when targets with different materials are used, the coating occurs simultaneously, in order to generate the above-described Ag islet structure.

Depending on the material of the surface structure, which is to be coated, a negative voltage of 100 V to 1500 V can be applied to said material in order to improve the adhesion and the layer homogeneity. To achieve the most uniform possible coating of the surface structure, the targets and the implant components can also be moved relative to one another during the coating process.

After the coating has occurred, and after a cooling phase, the coating chamber is ventilated again, and the coated implant component can be removed. The cooling can occur with the support of a gas atmosphere (for example, nitrogen or an inert gas) for improved heat removal, so that the cooling process is accelerated.

1. An implant component which comprises:
a solid material region;
a surface structure connected to the solid material region;
a coating which is provided on the surface structure, wherein the coating comprises an At % proportion of Ti as a main component, and Ag having an At % proportion of 15-25 At % as a further component; and
wherein the surface structure comprises undercuts which are coated with the coating.

2. The implant component according to claim 1, wherein the undercuts of the surface structure are at least partially formed by an open-pore structure.

3. The implant component according to claim 2, wherein the open-pore structure was generated by means of a plasma spray coating.

4. The implant component according to claim 2, wherein the open-pore structure comprises substantially regularly arranged unit cells and the unit cells are designed as tetrapod-like basic elements.

5. The implant component according to claim 2, wherein the open-pore structure has a porosity of 10% to 80% and/or a pore width of 45 μm to 1000 μm.

6. The implant component according to claim 1, wherein the surface structure is formed with a thickness of up to 4 mm, 3 mm, 2.5 mm, 2 mm, 1.5 mm, 1 mm or 0.5 mm.

7. The implant component according to claim 1, wherein the silver proportion of the coating is at least 18 At % and at most 25 At %.

8. The implant component according to claim 1, wherein the coating comprises an At % proportion of N and/or an At % proportion of Nb as a further coating component.

9. The implant component according to claim 1, wherein the is a Physical Vapor Deposition (PVD) coating.

10. The implant component according to claim 1, wherein the component Ag is present as islets in the coating.

11. The implant component according to claim 1, wherein the coating has a thickness of 1-6 μm.

12. The implant component according to claim 1, wherein the solid material region comprises an alloy comprising titanium.

13. A method for applying a coating onto an implant component, wherein the method comprises the steps of:
providing of an implant component of claim 1, wherein the implant component is formed with a solid material region and with a surface structure connected to the solid material region, and the surface structure comprises undercuts;
introducing of the implant component into a coating chamber;
providing of at least one target made of a metallic material which comprises at least one of the coated components;
locking of the coating chamber;
providing of an atmosphere with a pressure of 0.001 to 0.01 mbar;
igniting of an electric arc for evaporation of the metallic material of the at least one target; and
coating of the surface structure with the evaporated metallic material of the at least one target.

14. The method according to claim 13, wherein the surface structure is formed by particles melted to one another, which surface structure is applied by means of a plasma spraying method onto the solid material of the implant component.

15. The method according to claim 13, wherein the surface structure is constructed from unit cells and formed by means of a layer melting method.
